(19) United States
(12) Patent Application Publication
(10) Pub. No.: US 2021/0141452 A1
(43) Pub. Date: May 13, 2021

(54) VIRTUAL AND AUGMENTED REALITY GLOVE AND METHOD

(71) Applicant: HUMAN MODE, L.L.C., Oklahoma City, OK (US)

(72) Inventors: Jonathan Gilmore, Oklahoma City, OK (US); William X. Kerber, III, Edmond, OK (US)

(21) Appl. No.: 16/682,708

(22) Filed: Nov. 13, 2019

(51) Int. Cl. G06F 3/01; A41D 19/015; A61F 2/68; G06F 3/0346

(52) U.S. Cl. CPC G06F 3/014; G06F 3/016; G06F 3/0346; A41D 19/01588; A61F 2/68; G06F 3/017

(57) ABSTRACT

A sensory feedback system for managing and controlling the virtual interaction between a user and a central processing unit is provided. The sensory feedback system includes a central processing unit, a glove, a sensor system, and a microcontroller. The glove has a palm portion, a dorsal portion, and at least one finger member having a top side and a bottom side. The sensor system includes at least one flex sensor secured to the at least one finger member, a set of input and output sensors secured to the glove and the at least one finger member, and an inertial movement unit secured to the dorsal portion of the glove. The microcontroller is secured to the dorsal portion of the glove and facilitates transmission of signals between the sensor system and the central processing unit.

### TECHNICAL FIELD

The present disclosure relates generally to a sensory feedback system, and, more particularly, to a virtual and augmented reality glove design for receiving and transmitting signals between a user environment and a virtual or augmented reality environment and a method for the same.

### BACKGROUND

Virtual, augmented and mixed reality (collectively, “XR”) applications continue to evolve and become more capable everyday. Typically, the primary use for XR technology is in virtual reality gaming. As the capabilities of XR technology continue to evolve, humans spend more and more time playing, working, shopping and being entertained in XR environments. With continued evolution of XR technology, some common productivity applications or tasks may be more optimal, or even cost effective, to perform in the XR environment than they would be in the real world environment (“user's environment”). For example, training for high risk scenarios, such as surgery or military operations, artistic endeavors, such as painting or playing an instrument, or even remote monitoring of servers and robotics are all reasonable use cases for XR technology.

Maximizing the potential of XR technology platforms, whether for work or entertainment, requires optimal interfaces for the various tasks. One such interface is the human hand. The hand interacts and works with physical objects in the user's environment, so the hand provides an optimal interface to interact and work with virtual objects in the XR environment. The hands have great dexterity, accuracy, and sensitivity. The hands type, mix, wave, grab, perform sign language, and much more. This is why current XR technology platforms utilize hand controllers to interface between the XR environment and the user's environment.

Common hand controllers are hand-held controllers similar to those used for gaming consoles. Hand-held controllers tend to be bulky and more push button controllers rather than utilizing natural movement of the hand. Hand-held controllers provide limited accuracy when using individual fingers, which is necessary in order to successfully employ XR environments for productivity applications such as surgery or military operations which require precision accuracy. Without accurate tracking of finger position and movement, the dexterity, accuracy, and sensitivity of the hand is lost between the user's environment and the XR environment. These deficiencies in current hand-held controllers prompt the need for an XR glove controller design and method that accurately tracks and transmits not only hand position, but individual finger position and movement.

### SUMMARY

The present invention provides a sensory feedback system and method which overcomes the deficiencies described above, and has other advantages.

In one embodiment, a sensory feedback system is provided. The sensory feedback system comprises a central processing unit, a glove, a sensor system, and a microcontroller. The glove has a palm portion, a dorsal portion, and at least one finger member having a tope side and a bottom side. The sensor system comprises at least one flex sensor secured to the finger member, a set of input and output sensors, and an inertial movement unit secured to the dorsal portion of the glove. The set of input and output sensors comprises at least one of an input sensor. Alternatively, the set of input and output sensors may include an output sensor secured to the bottom side of the finger member and an input sensor or output sensor or both an input and output sensor secured to the palm portion of the glove. The microcontroller is secured to the dorsal portion of the glove and facilitates transmission of signals between the sensor system and the central processing unit.

In some embodiments, the sensory feedback system includes a light-emitting diode tracking system. The light-emitting diode tracking system comprises at least one light-emitting diode secured to the glove and at least one light sensor. The at least one light sensor receives a tracking input from the light-emitting diode and transmits the tracking input to the central processing unit.

Another embodiment provides a method for managing and controlling the virtual interaction between a user and a central processing unit. The virtual interaction method includes controlling the transmission of one or more signals between a glove and the central processing unit. The controlled signals provide interaction between a user environment and a virtual environment. Control of the signals between the glove and the central processing unit includes the steps: providing a microcontroller secured to the glove for facilitating the transmission of the one or more signals between the glove and the central processing unit; transmitting at least one flexure signal from the microcontroller to the central processing unit, wherein the flexure signal represents a degree of bend detected by a flex sensor secured to a finger member of the glove in the user environment; transmitting at least one impact signal from the microcontroller to the central processing unit, wherein the impact signal represents an impact in the user environment by at least one input sensor secured to the glove; transmitting at least one haptic signal from the central processing unit to the microcontroller, wherein the haptic signal represents an impact in the virtual environment by at least one output sensor secured to the glove; and transmitting at least one rotation signal from the microcontroller to the central processing unit, wherein the at least one rotation signal represents a detected rotation of the glove in at least one of the x, y, and z dimensions by an inertial movement unit secured to the glove.

In some embodiments, the method of managing and controlling the virtual interaction between the glove and the central processing unit may further comprise the steps of: transmitting at least one position signal from the microcontroller to the central processing unit, wherein the at least one position signal represents an identified position of the glove in a user environment by a light-emitting diode tracking system, the light-emitting diode tracking system comprising: at least one light-emitting diode secured to the glove; and at least one light sensor for receiving the position signal from the at least one light-emitting diode and transmitting the position signal to the central processing unit.

### BRIEF DESCRIPTION OF THE DRAWINGS

The drawings included with this application illustrate certain aspects of the embodiments described herein. However, the drawings should not be viewed as exclusive embodiments. In the Figures, the input and output sensors and light-emitting diodes are generically depicted as darkened circles, but the input and output sensors and the light-emitting diodes may take any configuration as suitable for use in connection with the various disclosed embodiments. The subject matter disclosed is capable of considerable modifications, alterations, combinations, and equivalents in form and function, as will occur to those skilled in the art with the benefit of this disclosure.

FIG. 1 is a block diagram of a sensory feedback system in accordance with one embodiment of the present disclosure.

FIG. 2 is a top view of the dorsal portion of a glove design in accordance with one embodiment of the present disclosure.

FIG. 3 is a bottom view of the palm portion of a glove design in accordance with one embodiment of the present disclosure.

FIG. 4 depicts both the palm portion and the dorsal portion of a glove having a light-emitting diode tracking system in accordance with one embodiment of the present disclosure.

FIG. 5 depicts both the palm portion and the dorsal portion of a glove having a light-emitting diode tracking system in accordance with one embodiment of the present disclosure.

FIG. 6 is a perspective view of a light-emitting diode tracking system defining a tracking space in accordance with one embodiment of the present disclosure.

FIG. 7 is a top perspective view of a glove having a light-emitting diode emitter pattern in accordance with one embodiment of the present disclosure.

FIG. 8 depicts a flow chart of the steps for managing and controlling the virtual interaction between a user and a central processing unit in accordance with one embodiment of the present disclosure.

### DETAILED DESCRIPTION

The present disclosure may be understood more readily by reference to these detailed descriptions. For simplicity and clarity of illustration, where appropriate, reference numerals may be repeated among the different figures to indicate corresponding or analogous elements. In addition, numerous specific details are set forth in order to provide a thorough understanding of the various embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Referring to FIGS. 1-7 generally, a sensory feedback system in accordance with the present disclosure is illustrated and generally designated by the numeral 10. Sensory feedback system 10 may be powered by AC/DC electricity or batteries. With reference to FIG. 1, the general form of sensory feedback system 10 includes a central processing unit 20, a glove 30, a sensor system 40, and a microcontroller 50. Sensory feedback system 10 may optionally include a light-emitting diode (“LED”) tracking system 60, as further depicted in FIGS. 4-7. The structure and use of central processing unit 20 are well known and will not be discussed in detail. Central processing unit 20 is selected for its ability to send to and receive signals from microcontroller 50 and LED tracking system 60. For example, in some embodiments, central processing unit 20 is an XR gaming console or an XR headset. In other embodiments, central processing unit 20 is a desktop computer.

As shown in FIGS. 2-7, glove 30 is a glove body for covering at least a portion of the hand. Glove 30 is datalinked to central processing unit 20 through microcontroller 50. For the purposes of this disclosure, “datalinked” means communication via wired or wireless connections such that signals can be received and transmitted between the linked components. The glove body has an inner surface and an outer surface. Glove 30 has a palm portion 32, a dorsal portion 34, and finger members 36. Finger members 36 have a top side 37A, a bottom side 37B, and a left and right side. The structure and use of a glove body to cover the hand are well known and will not be discussed in detail. Any conventional fabric that does not interfere with the operation of sensor system 40 may be used to construct glove 30. Such materials include but are not limited to: leather, vinyl, cotton and other fabrics. The type of glove 30 is selected for its ability to securely support sensor system 40. For example, sensor system 40 may be stitched or heat bonded to glove 30. In other embodiments, epoxy, glue, or resin may be used to secure sensor system 40 to glove 30.

In some embodiments, some or all of the components of sensor system 40 are secured to the outer surface of glove 30. In other embodiments, some or all of the components of sensor system 40 are secured to the inner surface of glove 30 such that sensor system 40 is not readily visible to the naked eye. In alternative embodiments, some components of sensor system 40 are secured to the outer surface of glove 30 and some components of sensor system 40 are secured to the inner surface of glove 30. In other embodiments, sensor system 40 is incorporated directly into the material comprising glove 30. For example, when incorporated directly into the material of glove 30, sensor system 40 may be positioned and secured between two pieces of material making up glove 30. As used herein, “secured” means attached to, carried by, or otherwise incorporated into.

Sensor system 40 includes one or more flex sensors 42, a set 44 of input and output sensors (referred to herein as set 44), and an inertial movement unit 46. As depicted in FIG. 3, a set of input and output sensors are identified by the bracketed element 44. Set 44 incudes a plurality of sensors 45a and 45b. Sensor system 40 is carried by a single glove 30. Thus, if a user is using two gloves 30, then the user is using two sensor systems 40. Each sensor system 40 is configured to interact with sensory feedback system 10. Each of flex sensors 42, set 44, and inertial movement unit 46 are datalinked to microcontroller 50. In addition, flex sensors 42, set 44, and inertial movement unit 46 may be datalinked to one another to allow for the transmission of signals.

Flex sensors 42 are secured to or incorporated into finger members 36 of glove 30. With reference to FIG. 2, flex sensors 42 are secured to top sides 37a of finger members 36. In other embodiments, flex sensors 42 are secured to bottom side 37b of finger members 36. Additionally, flex sensors 42 will also function if secured to either side of finger members 36. Although depicted in FIG. 2 as secured to each finger member 36, sensor system 40 will also function using only a single flex sensor 42 secured to a single finger member 36. In other embodiments, multiple numbers of flex sensor 42 may be incorporated into sensor system 40. Thus, sensor system 40 will function with any number of flex sensors 42 secured to a plurality of finger members 36. Typically, sensor system 40 will include five flex sensors 42, one flex sensor 42 secured to each finger member 36. Flex sensors 42 may extend along a portion of the length of finger members 36 or the entire length of finger members 36, extending from the knuckle portion 38 of glove 30 to the fingertip 37C of finger members 36.

Set 44 of input and output sensors is secured to glove 30 at various locations. Set 44 may include an input sensor 45a, an output sensor 45b, or a combination of the same. Typically, set 44 includes a plurality of input and output sensors 45a and 45b. As depicted in the Figures, input sensors 45a and output sensors 45b are depicted generically as darkened circles. However, it is to be understood that input sensors 45a and output sensors 45b may take any shape or geometric configuration.

Input sensors 45a register a user-actuated signal in the user environment for transmission as an input into the XR environment. Output sensors 45b receive a signal from the XR environment and generate a user-experienced sensation, output, in the user environment. In some embodiments, input sensor 45a or output sensor 45b is secured at fingertip 37C of bottom side 37b of finger member 36. In other embodiments, input sensor 45a or output sensor 45b is secured to palm portion 32 of glove 30. Additional embodiments may include input sensor 45a or output sensor 45b secured to joint 37d of bottom side 37b of finger members 36. In additional embodiments, input sensor 45a or output sensor 45b may be secured at each fingertip 37c of bottom side 37b of finger members 36, joint 37d of bottom side 37b of finger members 36, and to palm portion 32 of glove 30.

Input sensors 45a may be impact sensors. Types of impact sensors include, but are not limited to, piezoelectric, piezo film sensing material, force sensing resistors, or flat buttons. Piezo sensors are analog sensors suitable for creating a user perceived input, i.e., an impact experienced by the user in the real world. Such sensors require conversion from analog to digital form using an analog to digital converter. The structure and use of piezoelectric sensors are well known and will not be discussed in detail. Flat button sensors operate similar to keys on a keyboard in that they are user-actuated in the user environment. Flat button sensors register an input reflecting the application of pressure to the sensor, or the lack of pressure on the sensor. Force sensing resistors alter resistance when a force or pressure is applied.

Output sensors 45b may be haptic feedback sensors. Types of haptic feedback sensors include haptic motors such as small vibrational motors. Other haptic feedback sensors are possible, such as an Enamel insulated resistance heating wire or insulated Nichrome heating resistance wire (like those used in electric heating pads) could also be used to simulate touching objects of different warmth in the virtual environment. In some embodiments, where output sensors 45b and input sensors 45a are utilized, input sensors 45a may be secured to the fingertip 37c portions on bottom side 37b of finger members 36 and output sensors 45b may be secured at joint 37d distal from fingertip 37c.

Inertial movement unit 46 is secured at a central portion of glove 30. Inertial movement unit 46 may be secured to palm portion 32 or dorsal portion 34 of glove 30. Inertial movement unit 46 utilizes a combination of accelerometers and gyroscopes to track motion and orientation of glove 30 in the user's environment in three dimensions (x, y, and z). Inertial movement units 46 are commercially available as represented by Adafruit 9-DOF Absolute Orientation IMU Fusion Breakout—BNO055 and others known in the art.

In some embodiments, as depicted in FIGS. 4-7, sensory feedback system 10 includes LED tracking system 60 to measure positional data of glove 30 in the user environment. LED tracking system 60 is datalinked to central processing unit 20. LED tracking system 60 includes one or more LED emitters 62 and two or more light sensors 64. In most instances, in order to preclude accidental occlusion, sensory feedback system 10 includes at least three light sensors 64. LED emitters 62 are datalinked to light sensors 64.

LED emitters 62 are secured to glove 30. The structure and use of LED emitters 62 are well known and will not be discussed in detail. Any spectrum of light detectable by light sensors 64 may be utilized as LED emitters 62. However, it is generally preferable to use a non-visible light such as infrared for user convenience and comfort.

In some embodiments, as depicted in FIG. 4, LED emitters 62 are secured across the wrist portion of palm portion 32 and dorsal portion 34 of glove 30. In other embodiments, as depicted in FIG. 5, LED emitters 62 are secured along the length of top side 37a and bottom side 37b of one or more finger members 36. In additional embodiments, LED emitters 62 are secured across a combination of the wrist portion of palm portion 32 and dorsal portion 34 of glove 30 and up and down the length of top side 37a and bottom side 37b of one or more finger members 36.

In other embodiments, LED emitters 62 are secured to glove 30 in a pattern. For example, the pattern may be a straight line, a circle, a triangle, a square, or an arrow. The pattern may be the same or different on palm portion 32 and dorsal portion 34. Differing patterns allow for easier recognition between palm portion 32 and dorsal portion 34 by light sensors 64. For example, LED emitters 62 may be a circle in the center portion of palm portion 32 and a straight line across the knuckles portion 38 of dorsal portion 34. FIG. 7 depicts one example where LED emitters 62 correspond generally to the skeletal structure of the hand. In this example each fingertip 37c on top side 37a and on bottom side 37B and each joint 37d of finger members 36 on top side 37a and bottom side 37b carries LED emitters 62. The resulting configuration of LED emitters 62 provides a pattern on both palm portion 32 and dorsal portion 34 of glove 30.

Light sensors 64 positioned throughout the user's room and/or integrated into other XR technology worn or utilized by the user, monitor the position of the LED emitters 62 to define tracking space 66. For example, in some embodiments, the user's room may include light sensors 64 positioned in the corners of the room. In other embodiments, light sensors 64 are integrated into central processing unit 20. In additional embodiments, light sensors 64 are located adjacent to central processing unit 20. As depicted in FIG. 6, in some embodiments, light sensors 64 may be worn on a user's body through existing XR headsets or XR wearable tracking gear. Positioning of light sensors 64 is based on the orientation and visibility of LED emitters 62.

LED tracking system 60 includes a view field or tracking space 66 as defined by light sensors 64. With reference to FIG. 6, tracking space 66 is depicted linearly as projection lines from each light sensor 64. Invisible to the user's eye during operation, tracking space 66 provides a viewing field to track and measure LED emitters 62 within the user environment. Thus, the size of tracking space 66 depends on the number and positioning of light sensors 64. The size of tracking space 66 may vary depending upon the application of sensory feedback system 10. For example, for surgical training purposes, where sensory feedback system 10 controls a surgical machine, a smaller tracking space 66 may be utilized. In contrast, for artistic purposes, where sensory feedback system 10 controls a paintbrush, tracking space 66 may be much larger to allow for the use of a larger virtual canvas in the XR environment.

Microcontroller 50 is secured to glove 30. In some embodiments, microcontroller 50 is secured at a central portion 35 of dorsal portion 34 of glove 30. Microcontroller 50 is datalinked to sensor system 40 and central processing unit 20. Microcontroller 50, sensor system 40 and central processing unit 20 may be datalinked via wired or wireless connections. Microcontroller 50 communicates inputs and outputs to and from sensor system 40 and central processing unit 20.

Microcontroller 50 translates a user's action in the user environment to the XR environment and vice versa. For example, if a user makes a peace sign in the user environment, sensor system 40 sends an input to microcontroller 50 signaling the peace sign has been made. Microcontroller 50 transmits the input to central processing unit 20 which creates an output in the XR environment. In this example, the output in the XR environment may be an avatar in the XR environment making a peace sign. In another example, the output may control a real environment robotic hand driving the robotic hand to form the peace sign in the user environment. In an additional example, the user's avatar in the XR environment may create an input through impact with an object in the XR environment. The output may be output sensors 45B vibrating to signal the impact in the user environment.

As depicted in FIG. 8, another embodiment provides a method 100 of virtual interaction between a user and central processing unit 20. The virtual interaction includes the transmission of one or more signals between glove 30 and central processing unit 20. The signals provide interaction between the user environment and the XR environment. In step 102, microcontroller 50 is provided and secured to glove 30. Microcontroller 50 facilitates the transmission of one or more signals between glove 30 and central processing unit 20. In step 104, the microcontroller transmits at least one flexure signal to central processing unit 20. In step 106, microcontroller 50 transmits at least one impact signal to central processing unit 20. In step 108, central processing unit 20 transmits at least one haptic signal to microcontroller 50. In step 110, microcontroller 50 transmits at least one rotation signal to central processing unit 20.

The flexure signal represents the degree of bend detected by flex sensors 42. Flex sensors 42 measure the amount of deflection or bending of finger members 36. When a user bends a finger member 36, flex sensor 42 measures the degree of bend and transmits the flexure signal to microcontroller 50. Microcontroller 50 transmits the flexure signal to central processing unit 20 which configures the flexure signal into an output to provide an action within the XR environment or an action to perform a specific task by a robotic hand in the real world. For example, if a user bends finger members 36 in a gripping motion, microcontroller 50 transmits the flexure signal, i.e. the degree of bend of finger member 36, to central processing unit 20 which creates an output to cause an avatar in the XR environment to grip a virtual object. In another example, the output may be to grip an object in the user environment with a robotic hand.

The impact signal represents an impact in the user environment by at least one input sensor 45a. Input sensors 45a may be impact sensors which register an input, impact signal, when a user makes contact or impact with an object in the user environment such as tapping a table or clapping the user's hands together. Microcontroller 50 receives the impact signal and transmits the impact signal to central processing unit 20 to provide an action in the XR environment. The use of impact sensors as input sensors 45a allow for the exact timing of when contact or impact was made for accurate transmission to the XR environment. For example, if playing a virtual piano in the XR environment, using sensory feedback system 10, a user can play a piano in the user environment and input sensors 45a register the timing of the keys of the piano being played as an impact signal. Microcontroller receives the impact signal and transmits the impact signal to central processing unit 20. Central processing unit 20 then accurately reflects a user's impact with the piano in the user environment on the virtual piano in the XR environment.

The haptic signal represents an impact in the virtual environment by at least one output sensor 45b. Central processing unit 20 transmits a haptic signal received from the XR environment to microcontroller 50. Microcontroller 50 creates and translates the haptic signal into a haptic output. Microcontroller 50 transmits the haptic output to output sensors 45b which are haptic feedback sensors. When the haptic feedback sensors are haptic motors, the haptic output is a set vibration amount. The strength of the haptic output, i.e., vibration, will vary depending on the haptic signal created by the impact in the XR environment. For example, when playing a virtual piano in the XR environment, the haptic feedback sensors can register a set vibration amount when the piano key is touched in the XR environment based upon the density of the piano key in the XR environment that creates the haptic signal. Such vibration amount provides for a resistive sensation in the user environment providing the effect in the user environment that the piano key is resisting finger members 36.

The rotation signal represents a detected rotation of glove 30 in at least one of the x, y, and z dimensions. One or more accelerometers in inertial movement unit 46 detect the linear acceleration of glove 30 relative to an initial position of glove 30 as a user's hand moves in the user environment. For example, the one or more accelerometers in inertial movement unit 46 measure forward and back, up and down, and left and right movement of glove 30 in the user environment. One or more gyroscopes in inertial movement unit 46 detect the rotational rate of glove 30 relative to an initial position of glove 30 as a user's hand moves in the user environment. For example, the one or more gyroscopes of inertial movement unit 46 measure pitch, yaw, and roll of glove 30 in the user environment.

Inertial movement unit 46 receives measurements from the accelerometers and gyroscopes as raw data to generate a rotation signal. In some embodiments, the rotation signal is the estimated position of glove 30 in the user environment. Inertial movement unit 46 translates the raw data into an estimated position of glove 30 in the user environment. Inertial movement unit 46 transmits the rotation signal to microcontroller 50 which transmits the rotation signal, i.e., the position of glove 30, to central processing unit 20. Central processing unit 20 displays the rotation signal as movement or action in the XR environment. For example, the movement or action may be an avatar rotating its hand in the XR environment to open a door. In other embodiments, the rotation signal is the raw data from the accelerometers and gyroscopes. Inertial movement unit 46 transmits the rotation signal, i.e. the raw data from the accelerometers and gyroscopes, to microcontroller 50. Microcontroller 50 transmits the rotation signal to central processing unit 20 which estimates the position of glove 30 in the user environment. Central processing unit 20 displays the rotation signal as movement or action in the XR environment.

In some embodiments, the method for providing virtual interaction between a user and central processing unit 20 further comprises the steps of: transmitting at least one position signal of glove 30 to central processing unit 20. The position signal represents an identified position of glove 30 in the user environment by LED tracking system 60. Light sensors 64 identify light of a specific pattern and spectrum emitted from LED emitters 62 within tracking space 66 to create the position signal. Light sensors 64 transmit the position signal to central processing unit 20. The pattern of LED emitters 62 allows for light sensors 64 to detect light emitted from LED emitters 62 without regard to the position of the user's hand. For example, when LED emitters 62 are secured to palm portion 32 and dorsal portion 34 of glove 30, light sensors 64 detect light from LED emitters 62 regardless of whether the user's hand is facing upwards or downwards. Central processing unit 20 receives the position signal from light sensors 64 and estimates the position of glove 30 in the user environment.

Although the disclosed invention has been shown and described in detail with respect to a preferred embodiment, it will be understood by those skilled in the art that various changes in the form and detailed area may be made without departing from the spirit and scope of this invention as claimed. Thus, the present invention is well adapted to carry out the object and advantages mentioned as well as those which are inherent therein. While numerous changes may be made by those skilled in the art, such changes are encompassed within the spirit of this invention as defined by the appended claims.

1. A sensory feedback system comprising:
a central processing unit;
a glove, the glove having a palm portion, a dorsal portion, and at least one finger member, the at least one finger member having a top side and a bottom side;
a sensor system, the sensor system comprising:
at least one flex sensor secured to the at least one finger member;
a set of input and output sensors, wherein the set of input and output sensors comprises at least one input sensor and at least one output sensor, the set of input and output sensors configured to have:
the at least one input sensor or the at least one output sensor secured to the bottom side of the at least one finger member;
the at least one input sensor or the at least one output sensor secured to the palm portion of the glove; and
wherein the at least one output sensor is configured to receive a signal from the central processing unit and generate a user-experienced sensation;
an inertial movement unit secured to the dorsal portion of the glove; and
a microcontroller secured to the dorsal portion of the glove, wherein the microcontroller facilitates transmission of signals between the sensor system and the central processing unit.

2. The sensory feedback system of claim 1, wherein the at least one flex sensor is secured at a position selected from the group consisting of the top side of the at least one finger member, the bottom side of the at least one finger member, a left side of the at least one finger member, and a right side of the at least one finger member.

3. The sensory feedback system of claim 1, wherein a first set of signals is transmitted between the sensor system and the microcontroller and a second set of signals is transmitted between the microcontroller and the central processing unit.

4. The sensory feedback system of claim 1, further comprising:
a light-emitting diode tracking system, the light-emitting diode tracking system comprising:
at least one light-emitting diode secured to the glove; and
at least one light sensor for receiving a tracking input from the at least one light-emitting diode and transmitting the tracking input to the central processing unit.

5. The sensory feedback system of claim 4, wherein the at least one light-emitting diode and the at least one light sensor define a tracking space.

6. The sensory feedback system of claim 4, wherein the light-emitting diode tracking system tracks a position of the glove in a user environment and transmits the position of the glove to a virtual environment.

7. The sensory feedback system of claim 4, wherein the light-emitting diode tracking system comprises at least four light-emitting diodes and at least one light-emitting diode is secured to the top side of the at least one finger member, at least one light-emitting diode is secured to the bottom side of the at least one finger member, at least one light-emitting diode is secured to the dorsal portion of the glove, and at least one light-emitting diode is secured to the palm portion of the glove.

8. The sensory feedback system of claim 4, wherein the at least one light-emitting diode is secured to the glove to the top side of the at least one finger member, the bottom side of the at least one finger member, the dorsal portion of the glove, or the palm portion of the glove.

9. The sensory feedback system of claim 8, wherein the light-emitting diode tracking system comprises at least two light-emitting diodes.

10. The sensory feedback system of claim 9, wherein when the at least two light-emitting diodes are secured on the dorsal portion of the glove or on the palm portion of the glove, the at least two light-emitting diodes are arranged in pattern.

11. The sensory feedback system of claim 10, wherein the pattern is selected from the group consisting of a straight line, a circle, a triangle, a square, and an arrow.

12. The sensory feedback system of claim 10, wherein the light-emitting diode tracking system comprises at least four light-emitting diodes and at least two light-emitting diodes are secured to the dorsal portion of the glove and at least two light-emitting diodes are secured to the palm portion of the glove.

13. The sensory feedback system of claim 12, wherein the at least two light-emitting diodes secured on the dorsal portion of the glove are arranged in a first pattern and the at least two light-emitting diodes secured on the palm portion of the glove are arranged in a second pattern, and wherein the first pattern differs from the second pattern.

14. The sensory feedback system of claim 13, wherein the first pattern and the second pattern are selected from the group consisting of a straight line, a circle, a triangle, a square, and an arrow.

15. The sensory feedback system of claim 1, wherein the at least one flex sensor has a linear configuration and extends along at least a portion of the at least one finger member.

16. The sensory feedback system of claim 1, wherein the at least one flex sensor monitors a flexure of the at least one finger member and transmits the flexure of the at least one finger member to the microcontroller.

17. The sensory feedback system of claim 1, wherein the set of input and output sensors are selected from the group consisting of a haptic sensor and an impact sensor.

18. The sensory feedback system of claim 17, wherein the set of input and output sensors includes at least one of the haptic sensor and at least one of the impact sensor.

19. The sensory feedback system of claim 17, wherein the haptic sensor comprises a haptic motor, the haptic motor receives an input from a virtual environment and creates an output in a user environment, wherein the input is an impact with an object in the virtual environment and the output is a degree of vibration of the haptic motor in the user environment.

20. The sensory feedback system of claim 17, wherein the impact sensor is selected from the group consisting of buttons, force sensing resistors, piezoelectric sensors, and piezoelectric film sensing material.

21. The sensory feedback system of 17, wherein the impact sensor receives an input from a user environment and creates an output in a virtual environment, wherein the input is an impact with an object in the user environment and the output is a response in the virtual environment.

22. The sensory feedback system of claim 1, wherein the inertial movement unit detects rotation of the glove in the x, y, and z dimensions.

23. The sensory feedback system of claim 1, wherein the inertial movement unit is secured to a central portion of the dorsal portion of the glove.

24. A method for managing and controlling the virtual interaction between a user and a central processing unit, the virtual interaction method includes controlling the transmission of one or more signals between a glove and the central processing unit, wherein the controlled one or more signals provide interaction between a user environment and a virtual environment, the method of controlling the one or more signals between the glove and the central processing unit comprising:
providing a microcontroller secured to the glove for facilitating the transmission of the one or more signals between the glove and the central processing unit;
transmitting at least one flexure signal from the microcontroller to the central processing unit, wherein the flexure signal represents a degree of bend detected by a flex sensor secured to a finger member of the glove in the user environment;
transmitting at least one impact signal from the microcontroller to the central processing unit, wherein the impact signal represents an impact in the user environment by at least one input sensor secured to the glove;
transmitting at least one haptic signal from the central processing unit to the microcontroller, the haptic signal representing an impact in the virtual environment by the user, wherein the microcontroller translates the haptic signal into a haptic output that generates a user-experienced sensation in the user environment by at least one output sensor secured to the glove; and
transmitting at least one rotation signal from the microcontroller to the central processing unit, wherein the at least one rotation signal represents a detected rotation of the glove in at least one of the x, y, and z dimensions by an inertial movement unit secured to the glove.

25. The method of claim 24 further comprising:
transmitting at least one position signal of the glove to the central processing unit, wherein the at least one position signal represents an identified position of the glove in a user environment by a light-emitting diode tracking system, the light-emitting diode tracking system comprising:
at least one light-emitting diode secured to the glove; and
at least one light sensor for receiving the position signal from the at least one light-emitting diode and transmitting the position signal to the central processing unit.
